(19) United States
(12) Patent Application Publication
(10) Pub. No.: US 2004/0059430 A1
(43) Pub. Date: Mar. 25, 2004

(54) Collagen-based biomaterial for tissue repair

(76) Inventors: Tae-Woon Kim, Seoul (KR); Sung-Young Park, Dong-gu (KR); Ho-Chan Hwang, Seoul (KR)

Correspondence Address: KNOBBE MARTENS OLSON & BEAR LLP, 2040 MAIN STREET, FOURTEENTH FLOOR, IRVINE, CA, 92614, US

(21) Appl. No.: 10/618,447

(22) Filed: Jul. 10, 2003

Related U.S. Application Data
(63) Continuation of application No. PCT/KR02/01679, filed on Sep. 5, 2002.

(30) Foreign Application Priority Data
Sep. 5, 2001 (KR) 2001/54489
Sep. 13, 2001 (KR) 2001/56337

(51) Int. Cl. A61F 2/02

(52) U.S. Cl. 623/23.72; 8/94.11

(57) ABSTRACT

The present invention relates to a process for preparing a biomaterial for tissue repair, which comprises the steps of cross-linking collagen of a collagen-based tissue obtained from a mammal, decellularizing the tissue and freeze-drying the cell-free tissue by employing a cryoprotective solution, and a biomaterial for tissue repair prepared by the said process. The process for preparing a biomaterial for tissue repair of the invention comprises the steps of procuring a collagen-based biological tissue from a mammal; treating the biological tissue with polyepoxy compound to obtain a biological tissue with cross-linked collagen structure; decellularizing the biological tissue thus obtained to give a cell-free tissue; and, immersing the cell-free tissue in a cryoprotective solution containing hyaluronic acid and freeze-drying the said tissue. In accordance with the present invention, a biomaterial for tissue repair with more stabilized collagen structure can be prepared by a simpler process than the prior processes, which makes possible the economical preparation of various biomaterials for tissue repair.

### RELATED APPLICATIONS

This application is a continuing application under 35 U.S.C. § 365 (c) of PCT Application No. PCT/KR02/01679 designating the United States, filed Sep. 5, 2002. The PCT Application was published in English as WO 03/024496 A1 on Mar. 27, 2003, and claims the benefit of the earlier filing date of Korean Patent Application Nos. 2001/54489, filed Sep. 5, 2001 and 2001/56337, filed Sep. 13, 2001. The contents of the Korean Patent Application Nos. 2001/54489 and 2001/56337, and the international application No. PCT/KRO2/01679 including the publication WO 03/024496 A1 are incorporated herein by reference in their entirety.

### BACKGROUND OF THE INVENTION

1. Field of the Invention

The present invention relates to a process for preparing a biomaterial for tissue repair, more specifically, to a process for preparing a biomaterial for tissue repair, which comprises the steps of cross-linking collagen of a collagen-based tissue obtained from a mammal, decellularizing the tissue and freeze-drying the cell-free tissue by employing a cryoprotective solution, and a biomaterial for tissue repair prepared by the said process.

2. Description of the Related Technology

A variety of injectable materials have been used as repair materials for the soft tissue and the dermal tissue to cure various dermatological diseases, such as malformed contour of face, injury or subsidence of the soft tissue due to trauma, and the stunted soft tissue. Representative examples of repair materials are liquid silicone, bovine collagen, and autologous skin or fat:

Among these ones, liquid silicone was used primarily in the army during World War II. Since medical grade liquid silicone, ‘360’, was developed for humans in the United States in 1963, it has been used actively as a repair material for transplantation in the early stage because of the over-lasting effect in the human body. However, ‘360’ is proven to be less satisfactory in the sense that it causes the inflammation, induration, discoloration, ulceration, migration, silicone granulomas and so on (see: Klein A. W., Rish D. C., J. Dermatol. Surg. Oncol., 11:337-339, 1985; Nosanchuk J. S., Arch. Surg., 97:583-585, 1968; Piechotta F. U., Aesthetic Plast. Surg., 3:347-355, 1979; Spira M., Rosen T., Clin. Plast. Surg., 20:181-188, 1993), which prevents it from becoming popular material for transplantation.

On the other hand, it has been known that bovine collagen requires the sensitivity test on the skin of recipients before transplantation. Moreover, about 3% of transplantation recipients showed the hypersensitivity, even though they were normal in the sensitivity test checked before transplantation (see: Elson M. L., J. Am. Acad. Dermatol., 18:707-713, 1998), and the lasting period of transplantation was relatively short, from three months to six months (see: Gromley D. E., Eremia S., J. Dermatol. Surg. Oncol., 16:1147-1151, 1990; Matti B. A., Nicolle F. V., Aesthetic Plast. Surg., 14:227-234, 1990). In addition, it has been also reported that bovine collagen causes several transient side effects after transplantation, such as erythema, swellings, local necrosis of skin, and abscess (see: Cooperman L. S. et al., Aesthetic Plast. Surg., 9:145-151, 1985; Frank D. H. et al., Plast. Reconstr. Surg., 87:1080-1088, 1991; Hanke C. W., et al., J. Am. Acad. Dermatol., 25:319-326, 1991; Matti B. A. et al., Aesthetic Plast. Surg., 14:227-234, 1990).

Alternatively, autologous skin has been used in the art since it is relatively safe and does not require the sensitivity test and the lasting period of transplantation is from one year to two years. However, it has shortcomings that the excision of autologous skin requires long period of convalescence due to a complication of infection, and makes visible injuries on the body from which the skin is excised.

Finally, the use of autologous fat has increased with the progress of lipectomy, but it requires continuous transplantations to cure to the desired level since the lasting period of autologous fat is shorter than that of bovine collagen (see: Gromley D. E., Eremia S., J. Dermatol. Surg. Oncol., 16:1147-1151, 1990).

Each repair material described above has various merits for tissue repair in one or more aspect, but no material has satisfied the requirements of ideal repair materials for soft tissue.

Tissue bioengineering, including biomaterials art, has dramatically developed to overcome the above disadvantages, and some techniques have been utilized and commercialized in the art. In the near future, ideal repair materials would be introduced to substitute for soft tissue and the dermal tissue. However, all defects of repair materials described aboves cannot be solved by the conventional techniques and there is a great demand for the continuous development of relevant techniques. U.S. Pat. No. 5,336,616 discloses a method for producing an acellular collagen-based tissue for transplantation, which comprises the steps of removing antigenic cells inducing immune-rejection from the tissue and treating the tissue with a cryoprotective solution to reduce the damage of collagen structure in the course of freeze-drying. The acellular collagen-based tissue became popular, since it does not induce graft-rejection and it can be stored for a long time before use. The said method is, however, not satisfactory in light of the economy of production cost and the high feasibility to contamination. In addition, it has revealed a serious problem that collagen tissue would be damaged and rapidly degraded after transplantation, due to lack of a step of protecting the structure of collagen tissue prior to freeze-drying.

Under the circumstances, there are strong reasons for exploring and developing a simple and efficient process for preparing new biomaterial for tissue repair that causes little damage to the structure of collagen tissue.

### SUMMARY OF THE INVENTION

The present inventors have made an effort to manufacture a novel biomaterial for tissue repair in a simple and efficient process that causes little damage to the structure of collagen tissue.

An aspect of the present invention provides a biomaterial, which comprises: a collagen-based biological tissue from a mammal; and a plurality of cross-linking bonds between the tissue and one or more polyepoxy compounds. The biomaterial is substantially decelluralized. The biomaterial is substantially free from cells of the mammal. The biomaterial is substantially free from debris of cells of the mammal. A surface of the biomaterial is coated with a cryoprotective material. The biomaterial is in a freeze-dried form. The collagen-based biological tissue is from fascia, amnion, placenta or skin of a mammal. The one or more polyepoxy compounds comprise a backbone of 17-25 carbon atoms and 4-5 epoxy groups. The one or more polyepoxy compounds are selected from the group consisting of polyglycerol polyglycidyl ether, polyethylene glycol glycidyl ether and a mixture of the foregoing. The tissue comprises a helical structure of polypeptides. The plurality of cross-linking bonds are between the one or more polyepoxy compounds and one or more amino acids of the tissue. The biomaterial is in the form of powder. The collagen-based biological tissue comprises a bovine placental tissue or porcine skin tissue.

Another aspect of the present invention provides a method of using a biomaterial. The method comprises: providing the above-described biomaterial; and applying the biomaterial to a human or animal body part in need thereof. The biomaterial is in a powder form. The powder has a size from about 100 μm to about 500 μm. The application of the biomaterial comprises injecting into the body party a mixture comprising the powder in a liquid. The powder in the mixture has a concentration of from about 400 mg/ml to about 500 mg/ml. The liquid is PBS.

Another aspect of the present invention provides a method of providing a biomaterial. The method comprises: providing a collagen-based biological tissue from a mammal; and cross-linking the tissue using one or more polyepoxy compounds. The method further comprises removing cells from the tissue. The method further comprises destroying cells from the tissue. The method further comprises removing debris of the destroyed cells from the tissue. The method further comprises freeze-drying the tissue after the removal of cells. The method further comprises pulverizing the freeze-dried tissue. The pulverization is conducted in a pulverizer under an environment of liquid nitrogen. The method further comprises hydrating the freeze-dried tissue. The method further comprises cutting the hydrated tissue. The method further comprises coating a cryoprotective material over the tissue after the removal of cells.

In the above-described method, the cryoprotective material comprises hyaluronic acid. The collagen-based biological tissue is fascia, amnion, placenta or skin of a mammal. The one or more polyepoxy compounds comprise a backbone of 17-25 carbon atoms and 4-5 epoxy groups. The one or more polyepoxy compounds are polyglycerol polyglycidyl ether, polyethylene glycol glycidyl ether or a mixture of the foregoing. The tissue comprises a helical structure of polypeptides. The polyepoxy compound reacts with one or more amino acid to form a cross-linking bondage. The cross-linking comprises treating the biological tissue with 1-7% (w/v) of the one or more polyepoxy compounds. The cross-linking comprises treating the biological tissue with the one or more polyepoxy compounds at a pH from about 8 to about 11. The cross-linking comprises treating the biological tissue with the one or more polyepoxy compounds at a temperature from about 30 to about 45° C. The cross-linking comprises treating the biological tissue with the one or more polyepoxy compounds for about 10 to 20 hours.

Still another aspect of the present invention provides a biomaterial for tissue repair. The biomaterial is produced by the above-described method.

### BRIEF DESCRIPTION OF THE DRAWINGS

The above objects and features of the present invention will become apparent from the following descriptions given in conjunction with the accompanying drawings, in which:

FIG. 1 is a graph showing the comparison of cross-linking index at various temperatures.

FIG. 2 is a graph showing the comparison of cross-linking index at various concentrations of polyepoxy compounds.

FIG. 3 is a graph showing the comparison of cross-linking index at various pHs.

FIG. 4 is a graph showing the degradation of dermal layers by collagenase.

FIG. 5 is a graph showing the size of powder ground by various methods of pulverization.

FIG. 6 is a graph showing the lasting time in the subcutaneous layer of mice depending on the size of biomaterial for tissue repair and injection concentrations.

FIG. 7a is a photograph showing the dermal tissue one week after subcutaneous injection.

FIG. 7b is a photograph showing the dermal tissue one month after subcutaneous injection.

FIG. 7c is a photograph showing the dermal tissue one year after subcutaneous injection.

### DETAILED DESCRIPTION OF THE PREFERRED EMBODIMENT

The process for preparing a biomaterial for tissue repair of the invention comprises the steps of: procuring a collagen-based biological tissue from a mammal; treating the biological tissue with polyepoxy compound to obtain a biological tissue with cross-linked collagen structure; decellularizing the biological tissue thus obtained to give a cell-free tissue; and, immersing the cell-free tissue in a cryoprotective solution containing hyaluronic acid and freeze-drying the said tissue. The collagen-based tissue includes, but not limited these to, preferably fascia, amnion, placenta or skin of mammals. Polyepoxy compound includes, but not limited these to, preferably polyglycerol polyglycidyl ether, polyethylene glycol glycidyl ether, or other commercially available polyepoxy compounds. Preferably, 1-7% (w/v) of polyepoxy compound is treated on biological tissue at the condition of pH 8-11, 30-45° C. for 10-20 hours. Further, the freeze-dried cell-free tissue is preferably pulverized by physical means, for example, cryo-pulverization is carried out in a pulverizer under an environment of liquid nitrogen, to protect it from the damage by heat generated in the course of processing. The invented method may further comprise a step of pulverizing the freeze-dried cell-free tissue into smaller ones under an environment of liquid nitrogen before the cryo-pulverization or the steps of hydrating the freeze-dried cell-free tissue and cutting the hydrated tissue.

So far, a variety of cross-linking techniques have been developed to stabilize the structure of collagen, while maintaining the mechanical strength and unique properties of collagen tissues for transplantation. In addition to the cross-linking techniques, studies on decellularizing technique has been actively performed to reduce the immune-rejection against transplanted graft during transplantation, to proliferate cells in the graft and to develop new biomaterials for tissue engineering. Many researches related to glutaraldehyde have been conducted to increase the stability of tissue structure, which revealed a serious problem of the high toxicity of glutaraldehyde in human bodies. In this regard, alternative techniques for the cross-linking of collagen tissue have been explored in the art, one of which is cross-linking technique of collagen tissue using polyepoxy compounds.

Polyepoxy compounds have backbones of various lengths and functional groups. Commercially available Denacol™ EX-512 (Nagase Chemical Company, Japan) has been generally used for cross-linking of tissue.

Polyepoxy compounds are different from glutaraldehydes in terms of cross-linking reaction mechanism. Epoxy group of polyepoxy compound reacts highly with a variety of functional groups such as amino groups, carboxyl groups, hydroxyl groups, phenol groups and alcohol groups, whereas glutaraldehydes react only with f-amino groups of lysine residues in protein. In particular, polyepoxy compounds comprising backbone of 17-25 carbons and 4-5 epoxy groups show a high efficiency for the cross-linking of helical polypeptide molecules such as collagen.

Moreover, the toxicity of polyepoxy compounds is lower than that of glutaraldehyde, and the antigenicity or immune-response induction of tissues decreases in proportion to the reaction time, in case of reacting with helical polypeptide molecules such as collagen. Naturally, it shows relatively good biocompatibility (see: Lohre J. M. et al., Artif Organs, 16:630-633, 1992; Uematsu M. et al., Artif. Organs, 22:909-913, 1998).

Collagen fiber's structure contributes to physicochemical and bio-mechanical properties of collagen-based tissue. A collagen fiber contains collagen consisting of three polypeptides each of which is twisted one another to form a helical structure, and is stabilized through cross-linking by covalent bonds. One molecule of polyepoxy compound reacts with two or more amino groups of collagen to form cross-linking bondage, which provides the tensile strength and bio-safety of transplantable tissues. Transplanted collagen-based tissues are generally degraded by proteases of the recipient, however the cross-linking bondages protect the transplanted collagen-based tissues against the action of proteases.

Based on the theoretical knowledge, the present inventors added a step of cross-linking of collagen using a polyepoxy compound to minimize the damage of collagen structure, which is a defective point in the conventional method of producing an acellular collagen-based tissue for transplantation (see: U.S. Pat. No. 5,336,616). That is, collagen-based tissues are treated with polyepoxy compounds to form cross-linking bondages between collagen fibers or in collagen fibers before the decellularization, which, in turn, strengthens and stabilizes the structure of collagen-based tissues.

On the other hand, the decellularizing technique has been actively investigated to completely remove cells inducing immune-rejection, from the collagen-based tissues. The decellularizing technique is employed to remove whole cells by chemicals, enzymes or mechanical methods without loss of extracellular matrix component. The technique has been considered as a critical step in the development of a biomaterial for tissue repair, because regeneration of veins and remodeling of transplanted materials by cell division are more active in decellularized tissue which has preserved its own mechanical properties. In the decellularizing step, it is essential to remove completely the debris as well as cells to avoid immune-rejection after transplantation. Several methods have been used in the art, though detergents are preferably used for the decellularization of tissue, for example, ionic detergents such as sodium dodecyl sulfate (SDS) or non-ionic detergents such as Triton (Triton X-100), Tween (Tween 20, Tween 80) and NP (nonidet P-10, nonidet P-40) are used as the detergent.

Freeze-drying (or lyophilization) technique is used for preserving tissues without the damage of cells or tissues. Prior to the freeze-drying, tissues are first immersed in a cryoprotective solution for the protection of tissue against the freezing damage. A cryoprotective solution consists of buffer solution and cryo-dryprotectants, where buffer solution plays a role of maintaining ionic strength and osmotic pressure of a cryoprotective solution, and cryo-dryprotectants protect tissues against physical or chemical damage in the course of freeze-drying. Further, cryo-dryprotectants inhibit the collapse of tissues induced by the recrystallization of ice crystals during freeze-drying and increase the stability of tissues by way of elevating the glass transition temperature. During the drying step, if the temperature of tissue is higher than the glass transition temperature, ice crystals increase in size by the recrystallization, and consequently they cause damage to tissues. However, cryo-dryprotectants not only reduce damage to tissues to the minimum, but also shorten the drying time of tissues, because the ratio of glassy ice crystals or cubic ice crystals, which are less stable and smaller in size than hexagonal ice crystals, increases in frozen tissues due to the increased glass transition temperature by cryo-protectants.

As the cryo-dryprotectants, depending on their purpose of use, the combinations of materials such as DMSO (dimethysulfoxide), dextran, sucrose, propylene glycol, glycerol, mannitol, sorbitol, fructose, trehalose, raffinose, 2,3-butanediol, HES (hydroxyethyl starch), PEG (polyethylene glycol), PVP (polyvinyl pyrrolidone), proline, hetastarch and serum albumin are conventionally used in the art. The safety of the materials was proved for humans. However, they has revealed a shortcoming that the cost for the method of production is very high, since conditions of combinations are very complex.

In accordance with the present invention, hyaluronic acid is employed as a cryoprotectant to improve the stability of cell-free tissue for transplantation as well as the biocompatibility after its transplantation. Hyaluronic acid is a polysaccharide having a high reactivity with water molecule and an unbranched polysaccharide formed by bundles of D-glucuronic acid/N-acetyl-D-glucosamine disaccharide unit, and is rich in extracellular matrices of various tissues such as skin or cartilage.

Major functions of hyaluronic acid include space-filling, structure-stabilizing, cell-coating and cell-protecting. Hyaluronic acids form an integrated system with fibrous proteins in the extracellular matrix to provide the matrix with the properties of elasticity, viscosity, protection, lubricity and stabilization. In addition, high fluidity of hyaluronic acids plays an important role in the hydration of extracellular matrix and allows metabolites to diffuse rapidly at a relatively low concentration.

In the present invention, it was found that hyaluronic acid functions as a cryoprotectant by its own polysaccharide-structure, which, in turn, improves biocompatibility of cell-free tissue in the body of recipient after transplantation.

The present invention is further illustrated in the following examples, which should not be taken to limit the scope of the invention.

### EXAMPLE 1

### Determination of the Treatment Conditions of Polyepoxy Compound

Harvested porcine skin was kept at 4° C., in RPMI-1640 (13200-076, Gibco-BRL, USA) media containing 50 ng/ml amphotericin B (A-9528, Sigma, USA) and 1 mM EDTA. Then, the skin was cut into a piece of 1×2 cm² to prepare experimental samples. After incubation of the samples in a solution of 330 mM EDTA for 2 hours, the epidermal layer was taken off. Then, dermal layers were washed several times with PBS. Washed samples were treated with Denacol™ EX-512 (Nagase Chemical Company, Japan) at various concentrations, temperatures and pHs and then cross-linking indexes of samples were measured and compared with one another.

### EXAMPLE 1-1

### Measurement of the Cross-Linking Index at Various Temperatures

The experimental samples were incubated in 50 ml of 4% (w/v) Denacol™ EX-512 solution of pH 9.5 at 25° C. or 37° C., while shaking at 30±5 rpm. After 3, 6, 9, 12, 15, 18, or 24 hours of incubation, the amount of free amino groups was measured using the ninhydrin assay. Ninhydrin reacts with amino acids of collagen to develope bluish purple. Non-cross-linked samples are used as controls.

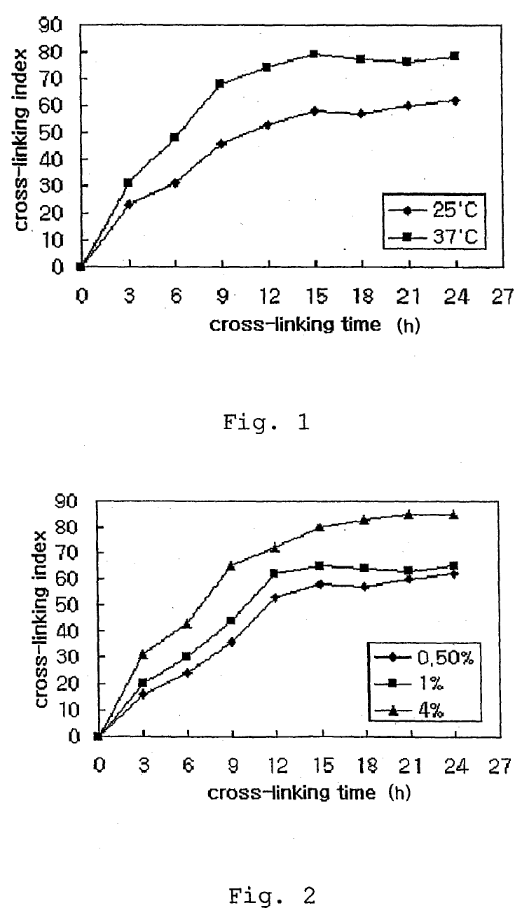

Samples obtained at the said incubation times were reacted with ninhydrin at 100° C. for 20 minutes and the absorbance was measured at 570 nm by the aid of spectrophotometer (Biomate 3, Thermo Spectronix). Different concentrations of N-6-acetyl lysine was used for the calibration of a standard curve and the value of mole conc. of collagen of samples against the mole conc. of free amine groups was considered as free amino groups. The cross-linking index was calculated according to the following equation (see: FIG. 1).

Cross-linking index=100×{1−(calculated value by ninhydrin)ₛₐₘₚₗₑ÷(calculated value by ninhydrin)_{control}}

FIG. 1 is a graph showing the comparison of cross-linking indexes at various temperatures. As shown in FIG. 1, it was demonstrated that: the cross-linking index increased in proportion to the reaction time until 9 hours and increased slightly after 9 hours; after 15 hours, the formation of cross-linking was observed; and, as the temperature increased, the cross-linking index began to increase.

### EXAMPLE 1-2

### Measurement of the Cross-Linking Index at Various Concentrations of Polyepoxy Compound

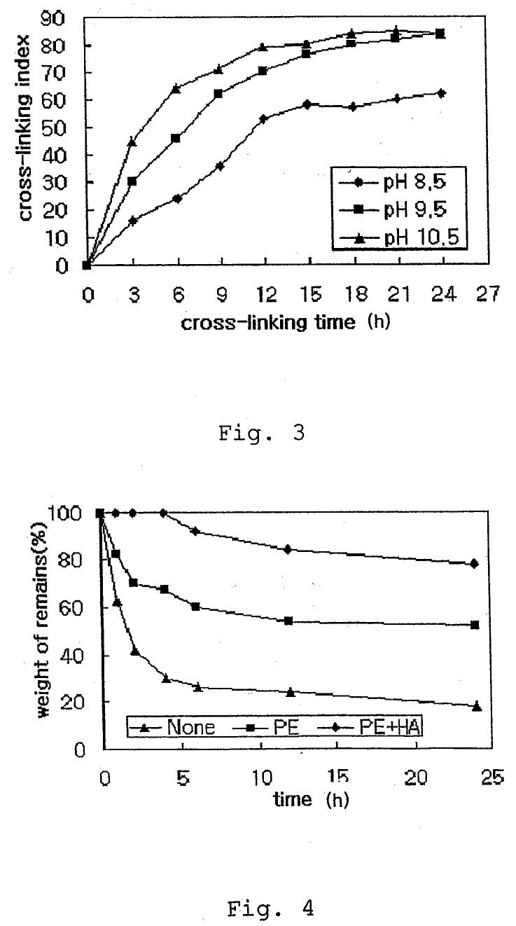

The experimental samples were incubated in 50 ml of 0.5, 1, and 4% (w/v) Denacol™ EX-512 solutions of pH 9.5 at 37° C., while shaking at 30±5 rpm. After 3, 6, 9, 12, 15, 18, and 24 hours of incubation, the amount of free amino groups was measured and the cross-linking index was calculated as described in Example 1-1 (see: FIG. 2). FIG. 2 is a graph showing the comparison of cross-linking index at various concentrations of polyepoxy compound. As shown in FIG. 2, the cross-linking index increased in proportion to the concentration of polyepoxy compound.

### EXAMPLE 1-3

### Measurement of the Cross-Linking Index at Various pHs

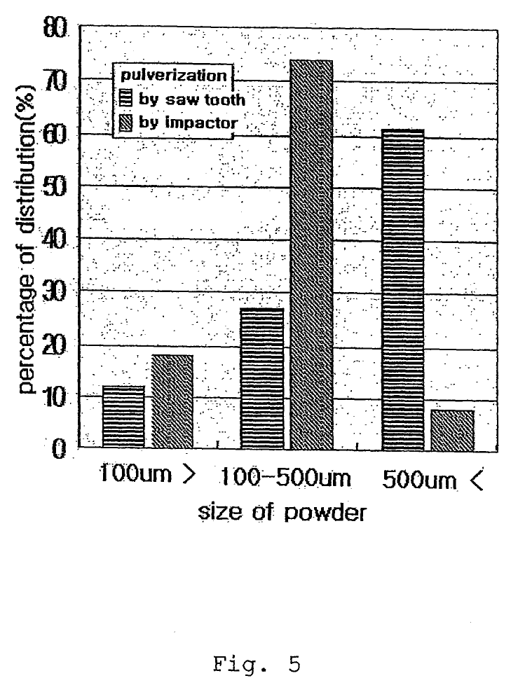

The experimental samples were incubated in 50 ml of 4% (w/v) Denacol™ EX-512 solutions of pH 8.5, 9.5, and 10.5 at 37° C., while shaking at 30±5 rpm. After 3, 6, 9, 12, 15, 18, and 24 hours of incubation, the amount of free amino groups was measured and the cross-linking index was calculated as described in Example 1-1 (see: FIG. 3). FIG. 3 is a graph showing the comparison of cross-linking indexes at various pHs. As shown in FIG. 3, it was found that as the value of pH increased, the cross-linking index began to increase.

From the above results, it was clearly demonstrated that the cross-linking of samples is optimized at the condition of 4% (w/v) polyepoxy compound, pH 9.5 and 37° C. of temperature.

### EXAMPLE 2

### Inhibitory Effect of Polyepoxy Compound and Hyaluronic Acid Treatment on Degradation of Collagen Structure

Harvested porcine skin was kept below 4° C., in RPMI-1640 media containing 5 μg/ml gentamicin (G-1397, Sigma, USA), 50 μg/ml amphotericin B and 1 mM EDTA. Then, the skin was placed dermal side down in a bioassay dish (Nalgene, USA) of 24.5×24.5 cm² and a corner of the skin was slit to identify the epidermal side and the dermal side. The skin was then cut into a rectangular piece of 6×10 cm² to prepare experimental samples. Samples were transferred to sterilized petri dishes (three samples per petri dish), and 50 ml of 0.5% protamine solution containing 330 mM EDTA was poured into each of petri dishes, and incubated at room temperature for 2 hours, while shaking at 45±5 rpm. Then, the epidermal layer and the dermal layer were separated by the aid of pincett. The dermal layers were washed several times with PBS, which were divided into three experimental groups as described now.

The first group (PE+HA) was incubated in 50 ml of 4% (w/v) Denacol™ EX-512 solution containing 1% (w/v) Tween 20 at the temperature of 37° C. for 15 hours, while shaking at 30±5 rpm and washed with PBS. Samples were incubated in 50 ml of 0.5% hyaluronic acid at 37° C. for 1 hour, while shaking at 30±5 rpm. After discarding hyaluronic acid solution, samples were washed with PBS and again incubated in 50 ml of 0.5% hyaluronic acid at 37° C. for 1 hour, while shaking at 30±5 rpm.

The second group (PE) was treated in a similar fashion as in the first group except for non-treatment of hyaluronic acid.

The third group (None) was incubated in 50 ml of 0.5% (w/v) SDS solution at room temperature for 12 hours and washed with PBS. Samples were then incubated in 50 ml of 10% (v/v) glycerol at room temperature for 2 hours.

After incubation of said experimental groups, samples were placed dermal side up in bioassay dishes. Bioassay dishes were put into a freeze-dryer (Ultra 35 super LE, Virtis, USA) that has a minimum shelf temperature of −50° C. and a minimum condenser temperature of −60° C. The samples were then frozen by rapidly decreasing the shelf temperature at a lowered rate of −2.5° C. per minute to −40° C. and left to stand for 10 minutes. The shelf temperature was then increased very slowly to reach to the temperature of 30° C. for 30 to 40 hours under a vacuum condition in order to dry the samples. The final moisture content of the dried samples is less than 5% (w/w). After drying, bioassay dishes were transferred to a laminar flow hood where the dried dermis was packed up by employing vacuum packing method and stored at 4° C.

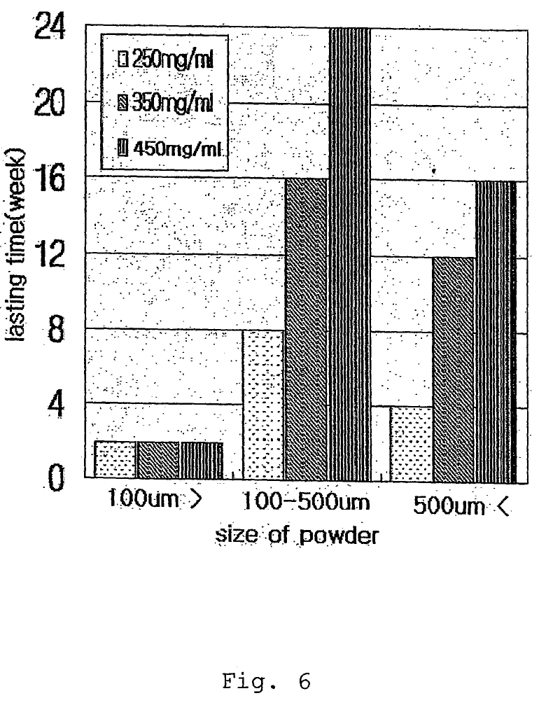

Freeze-dried samples of each group were cut into a piece of 1×3 cm², incubated in 10 mM CaCl₂ solution containing collagenase (1 U/ml) at 37° C. for a period of 25 hours, and collected at a time interval of 1 hour. The samples thus collected were weighed, and compared with the weight before the treatment of collagenase to analyze the levels of degradation of samples (see: FIG. 4). FIG. 4 is a graph showing the degradation of dermal layers by collagenase. As shown in FIG. 4, it was found that treatment of polyepoxy compound and hyaluronic acid efficiently reduced degradation by collagenase.

Consequently, it was clearly demonstrated that the dermal layers treated by polyepoxy compound and hyaluronic acid have more stable collagen structure than that prepared by the conventional method.

### EXAMPLE 3

### Preparation of a Biomaterial for Tissue Repair Using Bovine Placenta

Harvested bovine placental tissue was immediately placed in RPMI-1640 media containing 5 μg/ml gentamicin, 50 ng/ml amphotericin B and 1 mM EDTA and transferred to ice-packed container to keep the temperature below 4° C. until it was delivered to a clean bench. The delivered placental tissue was immersed in Dulbecco's phosphate-buffered saline (21600-010, Gibco-BRL, USA) containing 5 μg/ml gentamycin. The amnion was separated from placental tissue after discarding blood and the debris. The amnion was placed matrix side down in a bioassay dish (Nalgene, USA) and a corner of the tissue was slit to identify the epidermal side and the dermal side. The amnion was then cut into a rectangular piece of 6×10 cm² to prepare experimental samples. Samples were transferred to petri dishes (three samples per petri dish), and 50 ml of 0.5% protamine solution containing 330 mM EDTA was poured into each of petri dishes, and incubated at room temperature for 2 hours, while shaking at 45±5 rpm. Samples were then incubated in 50 ml of 4% (w/v) Denacol™ EX-512 solution containing 0.5% (w/v) SDS at the temperature of 37° C. for 15 hours, while shaking at 30±5 rpm, and washed again with PBS. The samples were incubated in 50 ml of 0.5% hyaluronic acid at 37° C. for 1 hour, while shaking at 30±5 rpm. After discarding hyaluronic acid solution, samples were washed with PBS and again incubated in 50 ml of 0.5% hyaluronic acid at 37° C. for 1 hour, while shaking at 30±5 rpm. The said samples were placed dermal side up in bioassay dishes and freeze-dried as described in Example 2.

### EXAMPLE 4

### Analysis of Distribution of Powder Size Depending on Pulverization Method

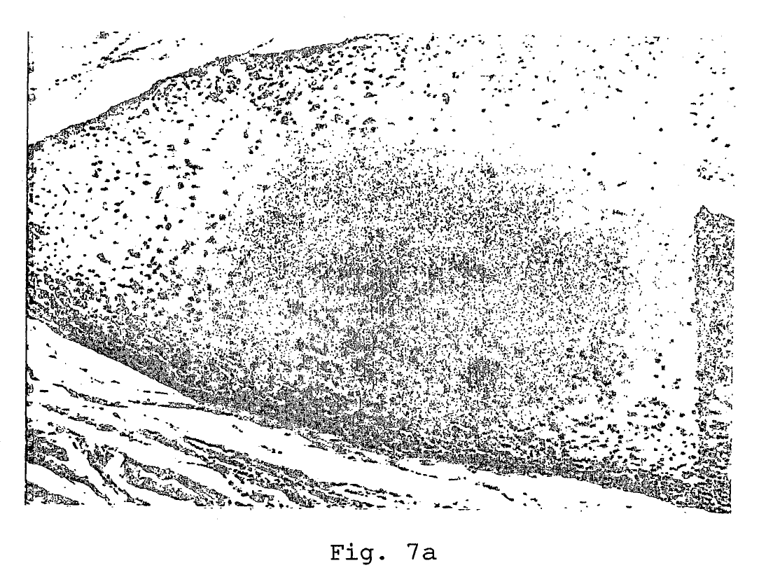

Two pulverization methods were employed to grind a biomaterial for tissue repair prepared in Example 3, to obtain fine injectable powder: The first one is performed by pulverizing 5 g of freeze-dried biomaterial for tissue repair by mechanical rotation of saw tooth equipped in a pulverizer under an environment of liquid nitrogen; and, the second one is performed by pouring 5 g of freeze-dried biomaterial for tissue repair in a sealed container and pulverizing by an impactor of the container equipped in a freezer mill (Freezer mill 6850, Spex CertiPrep, USA), while purging liquid nitrogen to the machine. The powder size of a biomaterial ground by the said two methods was compared with each other (see: FIG. 5). FIG. 5 is a graph showing the size of powder ground by the said two pulverization methods. As shown in FIG. 5, it was demonstrated that more than 70% of the biomaterial powder of 100-500 μm in size is obtainable by pulverization using a freezer mill by way of controlling the impact number of impactor, while more than 60% of the biomaterial powder of larger than 500 μm in size is obtainable by pulverization using a saw tooth whose rotation rate is uncontrollable.

### EXAMPLE 5

### Determination of Optimum Concentration of a Biomaterial for Transplantation

Subcutaneous transplantation was performed in 8 week-old male mice (Joongang Laboratory Animals Co. Ltd., Korea), to optimize the transplantation amount of a biomaterial powder prepared in Example 3.

A biomaterial powder was injected into the abdominal skin of mice anesthetized by ethyl ether in a clean bench, where experimental groups were divided into three groups by the size of powder, i.e., 100 μm>, 100-500 μm, 500 μm<, which was then divided into three groups by the concentration of powder, i.e., 250 mg/ml, 350 mg/ml, 450 mg/ml.

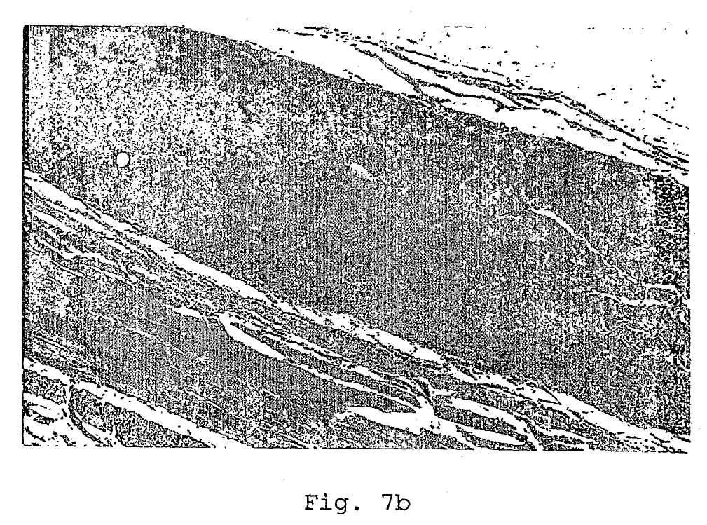

Injectable biomaterial for tissue repair was prepared by mixing each amount of the said powder with 1 ml of PBS in a leur-lok syringe. 0.5 ml of the mixture was injected subcutaneously into the abdomen using a 26-gauge needle. The lasting time of transplanted biomaterial was monitored with the naked eye at regular intervals (i.e., 1, 2, 4, 8, 12, 16, 20 and 24 weeks) during 24 weeks (see: FIG. 6). FIG. 6 is a graph showing the lasting time in the subcutaneous layer of mice depending on the size of biomaterial for tissue repair and injection concentrations. As shown in FIG. 6, it was examined that the lasting time of the biomaterial was the longest at 450 mg/ml regardless of the powder size and 100-500 μm in size showed the longest lasting time.

Consequently, it was clearly demonstrated that optimum size of powder was 100-500 μm and optimum concentration of powder was 450 mg/ml to maximize the lasting time of the biomaterial after transplantation.

### EXAMPLE 6

### Preparation of Biomaterial for Tissue Repair Using Porcine Skin and Transplantation

Harvested porcine skin was kept below 4° C., in RPMI-1640 media containing 5 μg/ml gentamicin, 50 ng/ml amphotericin B and 1 mM EDTA. Then, the skin was placed dermal side down in a bioassay dish and a corner of the skin was slit to identify the epidermal side and the dermal side. The skin was then cut into a rectangular piece of 6×10 cm² to prepare experimental samples. Samples were transferred to sterilized petri dishes (three samples per petri dish), and 50 ml of 0.5% protamine solution containing 330 mM EDTA was poured into each of petri dishes, and incubated at room temperature for 2 hours, while shaking at 45±5 rpm. Then, the epidermal layer and the dermal layer were separated by the aid of pincett. The dermal layers were washed with PBS and incubated in 50 ml of 4% (w/v) Denacol™ EX-512 solution containing 1% (w/v) Tween 20 at the temperature of 37° C. for 15 hours, while shaking at 30±5 rpm and washed again with PBS. Washed dermal layers were incubated in 50 ml of 0.5% hyaluronic acid at 37° C. for 1 hour, while shaking at 30±5 rpm. After discarding hyaluronic acid solution and washing with PBS, the dermal layers were again incubated in 50 ml of 0.5% hyaluronic acid at 37° C. for 1 hour, while shaking at 30±5 rpm. The said dermal layers were freeze-dried as described in Example 2. 4 g of freeze-dried biomaterial was ground into powder of 400 μm in size in a freezer mill as described in Example 4. Injectable biomaterial was prepared in an analogous manner as in Example 5, except for employing 1.5 ml of 1% (v/v) lidocaine solution instead of 1 ml of PBS.

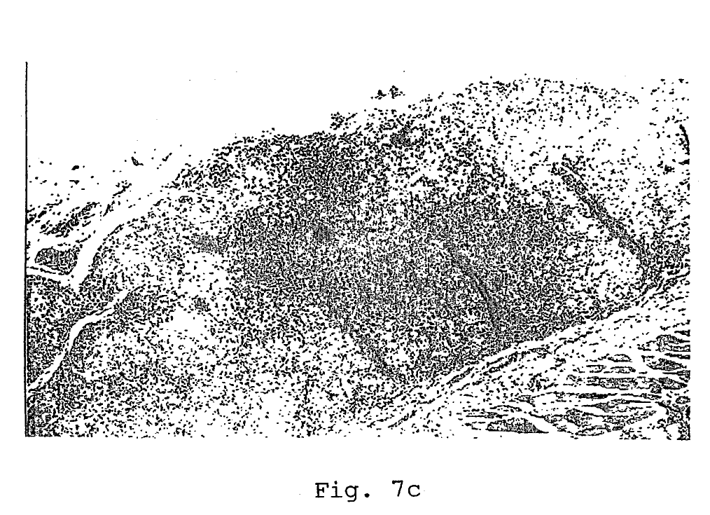

The injectable biomaterial thus prepared was injected to subcutaneous layers of male mice as described in Example 5. After 1 week, 1 month, and 12 months, the subcutaneous tissue was collected from the injected region and observed after staining with hematoxylin and eosin (H&E) to examine penetration and division of mouse cells (see: FIGS. 7a, 7b, and 7c). FIGS. 7a, 7b and 7c are photographs showing the dermal tissue one week after subcutaneous injection, one month after subcutaneous injection, and one year after subcutaneous injection, respectively. As shown in FIG. 7a, many mouse cells were penetrated and divided in the border of the injected region and a few cells started to divide in the center, after 1 week of transplantation; in FIG. 7b, mouse cells was actively divided in the center as well as in the border of the injected region, though the boundary of transplanted biomaterial and mouse tissue is obvious, and the transplanted biomaterial became autohistogenesis, after 1 month of transplantation; in FIG. 7c, mouse cells filled the injected region and the boundary disappeared to complete autohistogenesis, after 12 months of transplantation.

As illustrated and demonstrated above, the present invention provides to a process for preparing a biomaterial for tissue repair, which comprises the steps of crosslinking collagen of a collagen-based tissue obtained from a mammal, decellularizing the tissue and freeze-drying the cell-free tissue by employing a cryoprotective solution, and a biomaterial for tissue repair prepared by the said process. The process for preparing a biomaterial for tissue repair of the invention comprises the steps of procuring a collagen-based biological tissue from a mammal; treating the biological tissue with polyepoxy compound to obtain a biological tissue with cross-linked collagen structure; decellularizing the biological tissue thus obtained to give a cell-free tissue; and, immersing the cell-free tissue in a cryoprotective solution containing hyaluronic acid and freeze-drying the said tissue. In accordance with the present invention, a biomaterial for tissue repair with more stabilized collagen structure can be prepared by a simpler process than the prior processes, which makes possible the economical preparation of various biomaterials for tissue repair.

What is claimed is:

1. A biomaterial comprising:
a collagen-based biological tissue from a mammal; and
a plurality of cross-linking bonds between the tissue and one or more polyepoxy compounds.

2. The biomaterial of claim 1, wherein the biomaterial is substantially decelluralized.

3. The biomaterial of claim 1, wherein the biomaterial is substantially free from cells of the mammal.

4. The biomaterial of claim 1, wherein the biomaterial is substantially free from debris of cells of the mammal.

5. The biomaterial of claim 1, wherein a surface of the biomaterial is coated with a cryoprotective material.

6. The biomaterial of claim 1, wherein the biomaterial is in a freeze-dried form.

7. The biomaterial of claim 1, wherein the collagen-based biological tissue is from fascia; amnion, placenta or skin of a mammal.

8. The biomaterial of claim 1, wherein the one or more polyepoxy compounds comprise a backbone of 17-25 carbon atoms and 4-5 epoxy groups.

9. The biomaterial of claim 1, wherein the one or more polyepoxy compounds are selected from the group consisting of polyglycerol polyglycidyl ether, polyethylene glycol glycidyl ether and a mixture of the foregoing.

10. The biomaterial of claim 1, wherein the tissue comprises a helical structure of polypeptides.

11. The biomaterial of claim 1, wherein the plurality of cross-linking bonds are between the one or more polyepoxy compounds and one or more amino acids of the tissue.

12. The biomaterial of claim 1, wherein the biomaterial is in the form of powder.

13. The biomaterial of claim 1, wherein the collagen-based biological tissue comprises a bovine placental tissue or porcine skin tissue.

14. A method of using a biomaterial, the method comprising:
providing the biomaterial of claim 1; and
applying the biomaterial to a human or animal body part in need thereof.

15. The method of claim 14, wherein the biomaterial is in a powder form.

16. The method of claim 15, wherein the powder has a size from about 100 μm to about 500 μm.

17. The method of claim 15, wherein the application of the biomaterial comprises injecting into the body party a mixture comprising the powder in a liquid.

18. The method of claim 17, wherein the powder in the mixture has a concentration of from about 400 mg/ml to about 500 mg/ml.

19. The method of claim 17, wherein the liquid is PBS.

20. A method of providing a biomaterial, comprising:
providing a collagen-based biological tissue from a mammal; and
cross-linking the tissue using one or more polyepoxy compounds.

21. The method of claim 20, further comprising removing cells from the tissue.

22. The method of claim 20, further comprising destroying cells from the tissue.

23. The method of claim 22, further comprising removing debris of the destroyed cells from the tissue.

24. The method of claim 20, further comprising freeze-drying the tissue after the removal of cells.

25. The method of claim 24, further comprising pulverizing the freeze-dried tissue.

26. The method of claim 25, wherein the pulverization is conducted in a pulverizer under an environment of liquid nitrogen.

27. The method of claim 24, further comprising hydrating the freeze-dried tissue.

28. The method of claim 28, further comprising cutting the hydrated tissue.

29. The method of claim 20, further comprising coating a cryoprotective material over the tissue after the removal of cells.

30. The method of claim 29, wherein the cryoprotective material comprises hyaluronic acid.

31. The method of claim 20, wherein the collagen-based biological tissue is fascia, amnion, placenta or skin of a mammal.

32. The method of claim 20, wherein the one or more polyepoxy compounds comprise a backbone of 17-25 carbon atoms and 4-5 epoxy groups.

33. The method of claim 20, wherein the one or more polyepoxy compounds are polyglycerol polyglycidyl ether, polyethylene glycol glycidyl ether or a mixture of the foregoing.

34. The method of claim 20, wherein the tissue comprises a helical structure of polypeptides.

35. The method of claim 20, wherein the polyepoxy compound reacts with one or more amino acid to form a cross-linking bondage.

36. The method of claim 20, wherein the cross-linking comprises treating the biological tissue with 1-7% (w/v) of the one or more polyepoxy compounds.

37. The method of claim 20, wherein the cross-linking comprises treating the biological tissue with the one or more polyepoxy compounds at a pH from about 8 to about 11.

38. The method of claim 20, wherein the cross-linking comprises treating the biological tissue with the one or more polyepoxy compounds at a temperature from about 30 to about 45° C.

39. The method of claim 20, wherein the cross-linking comprises treating the biological tissue with the one or more polyepoxy compounds for about 10 to 20 hours.

40. A biomaterial for tissue repair produced by the method of claim 20.
